(19) United States
(12) Patent Application Publication
(10) Pub. No.: US 2024/0216167 A1
(43) Pub. Date: Jul. 4, 2024

(54) MODULAR BRACE SYSTEM

(71) Applicant: Sunbeam Products, Inc., Atlanta, GA (US)

(72) Inventors: Ryan Featherstone, Kalamazoo, MI (US); Sergiu Pop, Lake Worth, FL (US); Rebecca Wiener, Portage, MI (US)

(21) Appl. No.: 18/400,167

(22) Filed: Dec. 29, 2023

Related U.S. Application Data
(60) Provisional application No. 63/477,984, filed on Dec. 30, 2022.

(51) Int. Cl. A61F 7/02; A61F 5/01; A61F 5/02

(52) U.S. Cl. CPC A61F 7/02; A61F 5/0109; A61F 5/02; A61F 2007/0024

(57) ABSTRACT

The present disclosure relates generally to a modular brace system for either knee or back stabilization. The system includes either a knee brace or a back brace comprising a main body portion and side straps configured to wrap around a user's knee or back, as appropriate, and releasably secure to the brace to provide compression. The system also includes at least one of a gel pack and a heat pack to provide hot and/or cold therapy to the affected area. The gel and heat packs are releasably engageable with the brace so that they can be switched out as necessary.

### CROSS-REFERENCE TO RELATED APPLICATIONS

The present application claims priority to U.S. Provisional Patent Application No. 63/477,984, filed Dec. 30, 2022, the entire contents of which are hereby incorporated by reference.

### FIELD OF THE INVENTION

The present disclosure relates to a modular brace system for a knee brace or a back brace with integrations for hot and cold therapy.

### BACKGROUND OF THE INVENTION

Many people suffer from lower back or knee injuries and require a brace to help stabilize the injured area. The value of hot and cold therapy for easing and preventing the onset of pain in muscle tissue is also well established. However, when effecting hot and cold therapy, the injured area must be removed from the brace and thereby remove the stabilization. Furthermore, placing heating or cooling pads on the injured area typically restricts the user to an immobilized sitting or lying position.

### SUMMARY OF THE INVENTION

The present disclosure relates generally to a modular back brace or knee brace system that includes: (1) a brace for stabilization of a joint that is configured to hold one or more inserts using hook and loop material; (2) a cold gel pack insert designed to permit for hook and loop material to be attached and which is configured to attach to the brace; and (3) a heating pad insert that is also designed to permit for hook and loop material to be attached and configured to attach to the brace. In contrast to existing braces in which a traditional gel pack must be placed in a pouch for stability, through use of a gel pack laminated with fabric layers that allow stable attachment of hook and loop material, the current system uses a hook and loop material to create additional stability and flexibility for a consumer. Preferably, the hook and loop material is bonded to or sewn on the laminated fabric layers at multiple points, for example, at multiple corners of a rectangular pack.

The heating pad insert can be operated by a single push-button controller which can be placed in a designated location on the brace. The heating pad insert can be plugged directly into the wall with an adapter and extension cord or utilize a low voltage battery pack for portable therapy. The battery pack can be attached anywhere on the system via a clip design.

The braces described herein can deliver combination therapies that offer multiple benefits while also helping a user reduce the complexity and time needed to procure treatment. The braces also provide personalized pain relief in a modular system such that a user can choose and select the pieces of the system most useful to them. The present system includes a suite of products that work separately or together to deliver a complete and customized pain relief solution, allowing customizable therapies (portable hot or cold) with combined stabilization.

### BRIEF DESCRIPTION OF THE DRAWINGS

For a better understanding of the present invention, reference may be made to the following accompanying drawings. Corresponding reference numbers indicate corresponding parts throughout the drawings.

FIG. 1 is a rear perspective view of an embodiment of a back brace of the present disclosure.

FIG. 2 is a front perspective view of the back brace of FIG. 1.

FIG. 3 is a perspective view of an interior of the back brace of FIGS. 1 and 2, further illustrating a gel pack.

FIG. 4 is a perspective view of a heating pad for use with the back brace of FIGS. 1-3.

FIG. 5 is a perspective view of power cords for use with the heating pad of FIG. 4.

FIG. 6 is a perspective view of a battery pack for use with the heating pad of FIG. 4.

FIG. 7 is a perspective view of the back brace of FIGS. 1 and 2 with the battery pack of FIG. 6 in use.

FIG. 8 is a perspective view of the back brace of FIGS. 1 and 2, illustrating a cord management system thereof.

FIG. 9 is a front perspective view of an embodiment of a knee brace of the present disclosure.

FIG. 10 is a top plan view of an interior of the knee brace of FIG. 9, further illustrating a gel pack.

FIG. 11 is a perspective view of an interior of the knee brace of FIG. 9, further illustrating a heating pad.

FIG. 12 is a front perspective view of the knee brace of FIG. 9 with a battery pack in use.

FIG. 13 is a rear perspective view of the knee brace of FIG. 9, illustrating a cord management system thereof.

### DETAILED DESCRIPTION OF THE INVENTION

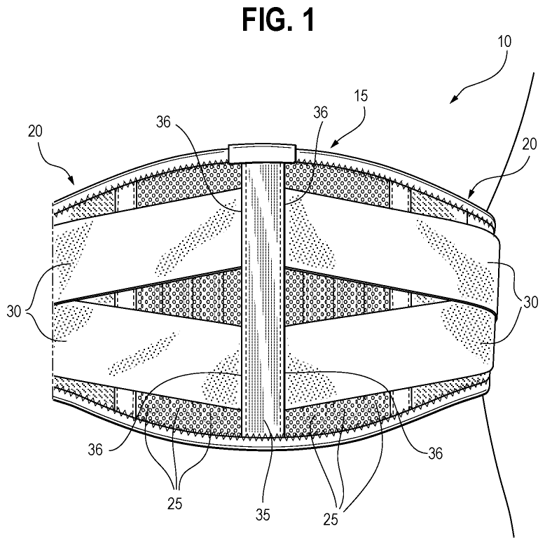

Turning now to FIG. 1, the present disclosure relates to a back brace, indicated generally by reference numeral 10. The back brace 10 can be worn around a user's core to allow for stabilization of the lower and mid-back. The back brace 10 can include a rear section 15 and side straps 20. The rear section 15 may be generally sized and shaped to correspond to a user's lower to mid-back and my taper inwardly towards the narrower side straps 20 in order to better contour the user's body profile. The side straps 20 are configured to meet and can overlap on a user's torso. The side straps 20 can include a releasable closure device, for example, hook and loop type fasteners, resilient snaps, or other similar fasteners to releasably fasten to each other. The position of the closure can be adjustable to allow for adequate compression and stabilization of users of different sizes.

The brace 10 can be formed from any appropriate material. In one embodiment, the rear section 15 includes mesh fabric. The rear section 15 of the brace 10 can also include stabilization rods 25 to help provide added support to the brace 10. In some embodiments, the stabilization rods 25 can be added into the interior of the material (e.g., mesh) of the rear section 15 so that they are not visible during standard use.

In order to provide added compression, the brace 10 can include secondary compression straps 30. The brace 10 can include one, two, three, four, or more compression straps 30. The illustrated embodiment includes four compression straps 30, two on each side of the brace 10. A first end 36 of the compression strap 30 can be captured by a central spine member 35 of the brace 10. The central spine member 35 can be made from a webbed material to allow for additional flex and compression.

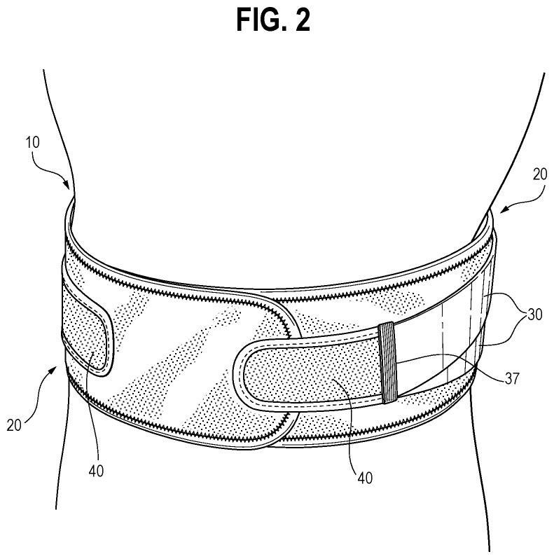

As illustrated in FIG. 2, a second end 37 of the compression strap 30 can be captured in an adjustable tab 40. The adjustable tab 40 can be coupled to one or more compression straps 30. The illustrated embodiment includes two adjustable tabs 40 on either side of the brace 10 each capturing both compression straps 30 on its respective side. The tabs 40 may or may not be attached to the side strap 20. When not attached, the tabs 40 include a releasable closure device, for example, hook and loop fasteners, resilient snaps, or other appropriate fasteners. Adjustment of the compression straps 30 can allow for additional compression benefits.

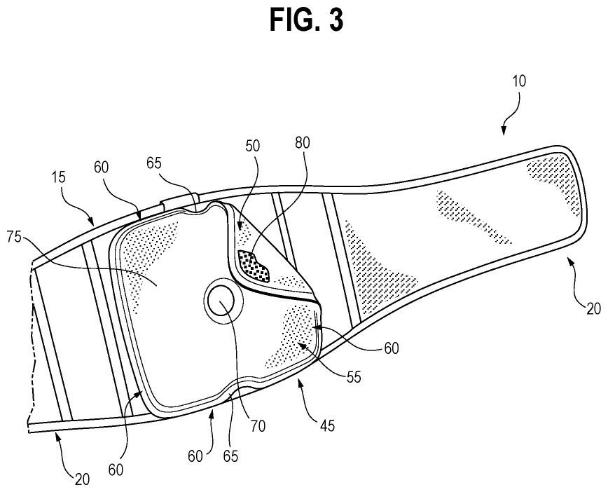

Turning now to FIG. 3, also provided herein is a gel insert 45 that is releasably insertable into the brace 10. In this way, the insert 45 is sized and shaped to be received on the interior side of the rear section 15 of the brace 10. The gel inserts 45 can include a first face 50 positionable against the interior of the rear section 15 of the brace 10 and a second face 55 opposing the first face 50 and positionable against a user's body. The first and second faces 50, 55 are joined by sides 60 (e.g., four sides 60 where the insert 45 is rectangular in shape, although other shapes, such as oval, square, circular, and the like are also foreseen). One or more sides 60 can include a cut-in or notch 65 to aid in the conformability of the insert 45 to body and brace 10 contours. The insert 45 can also include a central opening 70. In some embodiments, the opening 70 is circular.

The insert 45 can include an interior gel pack (not visible) and an exterior fabric barrier 75. In some embodiments, the gel pack is free-floating within the fabric barrier 75. The exterior fabric barrier 75 can allow for stitching of certain components (e.g., addition of fasteners) without puncturing the gel pack. Typically, the exterior fabric barrier is a laminated fabric barrier having two or more layers of fabric. In some embodiments, the fabric layers comprise the same type of fabric. In other embodiments, the types of fabric are mixed among the layers. For example, a first fabric layer can comprise foam backed brushed polyester, a second fabric layer can comprise brushed polyester, and the third fabric layer can comprise polyester mesh. Thus, the first face 50 of the insert 45 can include fasteners to releasably fasten the insert 45 to the interior of the brace 10. The illustrated embodiment includes hook and loop fastener fabric 80 on each of the four corners of the first face 50, although additional fastener types are foreseen. When hook and loop fasteners are employed, the hook and loop fastener fabric 80 of the insert 40 can be configured to engage with the fabric of the brace 10, or the brace 10 can include the corresponding hook and loop fastener fabric to engage the fabric 80.

The gel in the gel insert 45 is preferably both heatable and coolable. For example, the gel insert 45 can be placed in a freezer or refrigerator to freeze or cool the gel and provide cold therapy to the user. Alternatively, the gel insert 45 can be heated, for example, in the microwave, to provide hot therapy to the user.

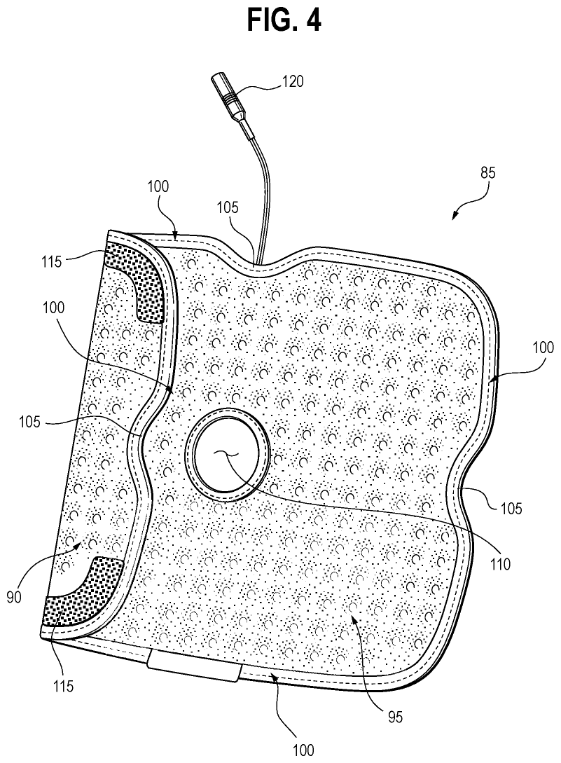

Referring now to FIG. 4, also provided herein is a heat insert 85 that is releasably insertable onto brace 10. In this way, the insert 85 can be sized and shaped to be received on the interior side of the rear section 15 of the brace 10. The heat insert 85 can include a first face 90 positionable against the interior of the rear section 15 of the brace 10 and a second face 95 opposing the first face 90 and positionable against a user's body. The first and second faces 90, 95 are joined by sides 100 (e.g., four sides 100 where the insert 85 is rectangular in shape, although other shapes, such as oval, square, circular, and the like are also foreseen). One or more sides 100 can include a cut-in or notch 105 to aid in the conformability of the insert 85 to body and brace 10 contours. The insert 85 can also include a central opening 110. In some embodiments, the opening 110 is circular.

The first face 90 of the insert 85 can include fasteners to releasably fasten the insert 85 to the interior of the brace 10. The illustrated embodiment includes hook and loop fastener fabric 115 on the four corners of the first face 90, although additional fastener types are foreseen. When hook and loop fasteners are employed, the hook and loop fastener fabric 115 of the insert 85 can be configured to engage with the fabric of the brace 10, or the brace 10 can include the corresponding hook and loop fastener fabric to engage the fabric 115.

The heating insert 85 can include interior electrical components to provide ambient heat through the insert 85 and to a user. Thus, the insert 85 can include an electrical cord connector 120 configured to couple the heating pack 85 to a power supply. The insert 85 can be constructed of or feature an insulating and/or reflective material in order to enhance the amount of ambient heat provided. For example, the insert 85 can include a reflective layer that can reflect heat radiated towards one side of the heating pad toward the other side so that less heat is lost to the ambient air. The insert 85 can further include a polyester batting fiber layer and a brushed polyester layer.

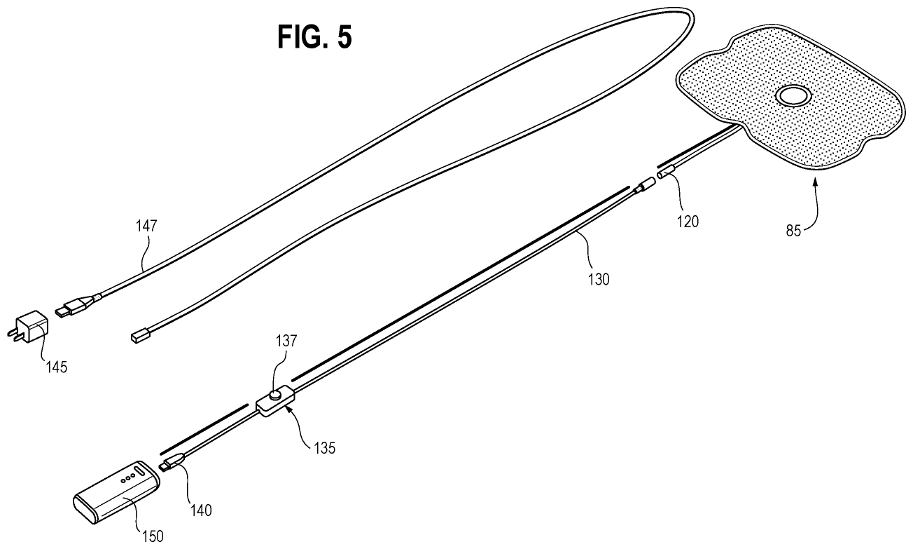

Turning now to FIG. 5, the electrical cord connector 120 is couplable to a controller cord 130, which is in turn electrically coupled to a controller 135. In one embodiment, the controller 135 can include a button or switch 137 that allows a user to turn the pack 85 on and off. The controller 135 can include an electrical port 140, for example, a USB (USB-A, USB-C, etc.) port. The port 140 can be a male port. The port 140 can be electrically connected to either (1) a power block 145 (in some embodiments, via an extension cord 147) to plug the heating pack 85 into an electrical outlet, or (2) to a battery pack 150.

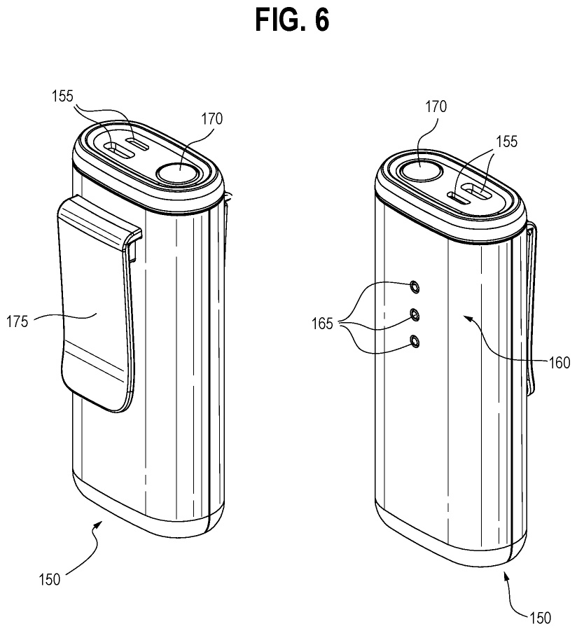

The battery pack 150 is shown in greater detail in FIG. 6. The battery pack 150 can include a female port 155 to receive the electrical port 140 of the controller 135. Some embodiments, such as the illustrated embodiment, may include multiple ports 155 to accommodate electrical ports 140 of varying sizes. Other devices (e.g., phones, electronic books, smart watches, etc.) can also be coupled to and charged by the battery pack 150 when an appropriate cord is coupled to the electrical ports 155 (e.g., via a USB-C 5V connector). The battery pack 150 can house an external power supply (e.g., a battery). The battery can be rechargeable, for example, by connecting the battery pack 150 to an electrical outlet via one of the female ports 155.

The battery pack 150 can include an indicator 160 that indicates to a user the power level remaining in the battery. The illustrated embodiment includes three LED lights 165 that function as the indicator 160. The number of LED lights 165 illuminated can indicate the power level. For example, when all three LEDs 165 are illuminated, this can indicate a battery level of 61% to 100%; when only two LEDs 165 are illuminated, this can indicate a battery level of 31% to 60%; when only one LED 165 is illuminated, this can indicate a battery level of 11% to 30%; when only one LED 165 and is blinking, this can indicate a battery level of less than 11%. It is understood systems with different numbers or arrangements of lights or the addition of sounds or haptics can also be used.

Typically, the LEDs 165 illuminate only when the controller 135 is in the ON position. The battery pack 150 can include an indicator button 170. When depressed, the indicator button 170 can trigger the LEDs 165 to temporarily illuminate (or to trigger another indicator 160) to indicate the battery pack 150 charge, even when the controller 135 is in the OFF position. The battery pack can also include a resilient clip 175.

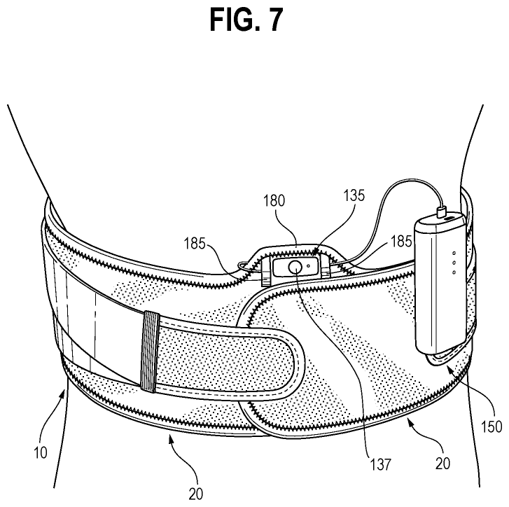

As illustrated in FIG. 7, the clip 175 (hidden from view) allows the battery pack 150 to be fastened onto the brace 10 to allow the user to freely move while using the heat pack 85 hands free. A front portion of a side strap 20 can include a controller projection 180 sized and shaped to receive the controller 135 thereon. The projection 180 can include at least one fastener 185 (for example, two fasteners in the illustrated embodiment). The fastener 185 can releasably fasten the controller 135 on the projection 180. In other embodiments, the controller 135 can have a releasable fastener on a portion thereof to releasably couple to the brace 10 (e.g., hook and loop type fasteners, resilient snaps, etc.).

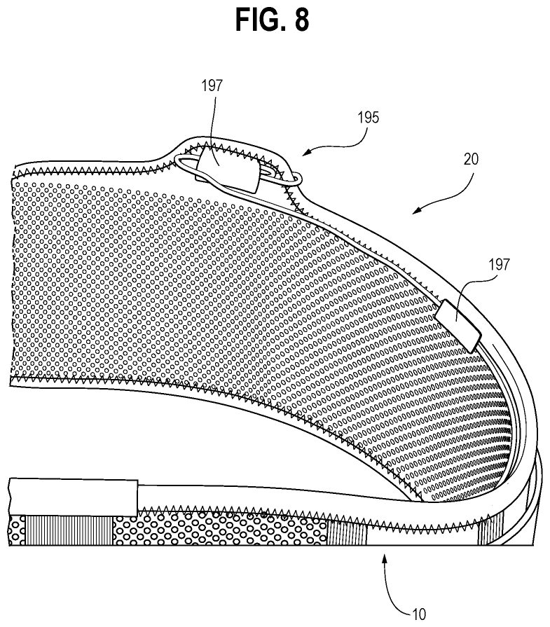

Turning now to FIG. 8, the brace 10 can include a cord management system 195. The cord management system can be disposed on either the interior or the exterior of the brace 10. The illustrated embodiment includes a cord management system disposed on the interior. The cord management system 195 can include any device to releasably secure the cord supplying power to the heating pack 85 to the brace 10, for example, clips, loops, or similar devices. The illustrated embodiment includes loops 197 through which the cord 130 can be fed. The loops 197 can be elastic loops. The cord management system 195 works to prevent the tangle of cords and cords getting in the way during use.

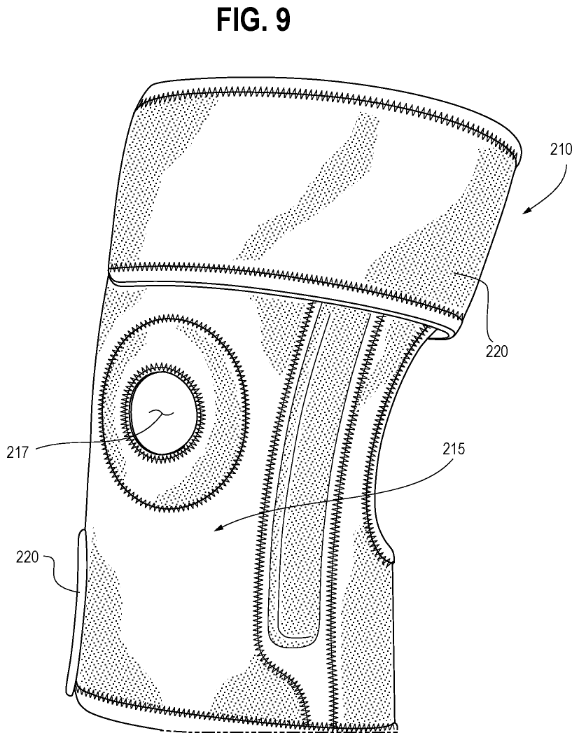

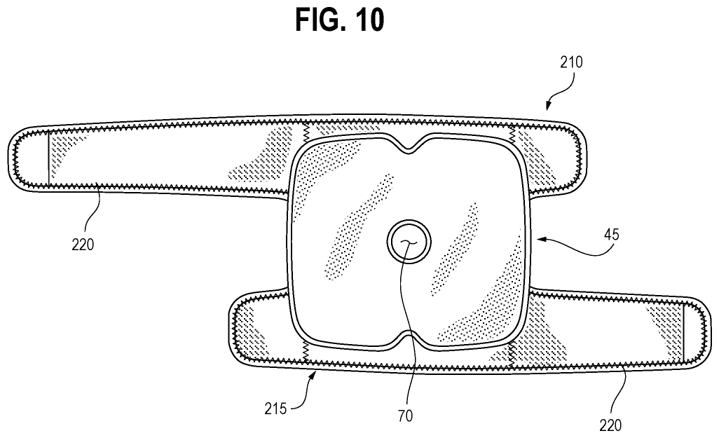

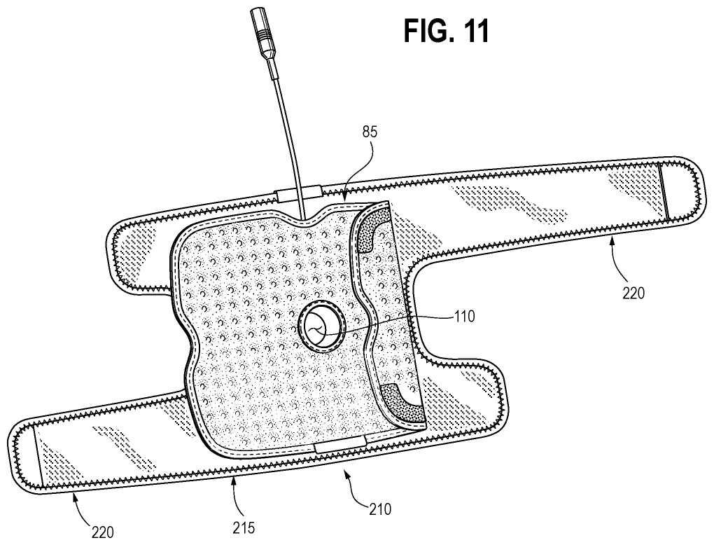

Referring now to FIG. 9, also provided herein is a knee brace generally indicated by reference numeral 210. Knee brace 210 is similar to back brace 10. The knee brace 210 includes a main body portion 215 and side straps 220. The side straps 220 wrap around a user's leg to secure the brace 220 to the user with compression. The side straps 220 releasably fasten to the main body portion 215 using, for example, hook and loop type fabric, although other types of releasable and adjustable closures are also foreseen. The main body portion 215 of the brace 210 can include a patella opening 217. In use, the patella opening 217 typically aligns with a user's patella. When the gel pack 45 (FIG. 10) or heat pack 85 (FIG. 11) include openings 70, 110 thereon, these openings can be sized and shaped to align with the patella opening 217.

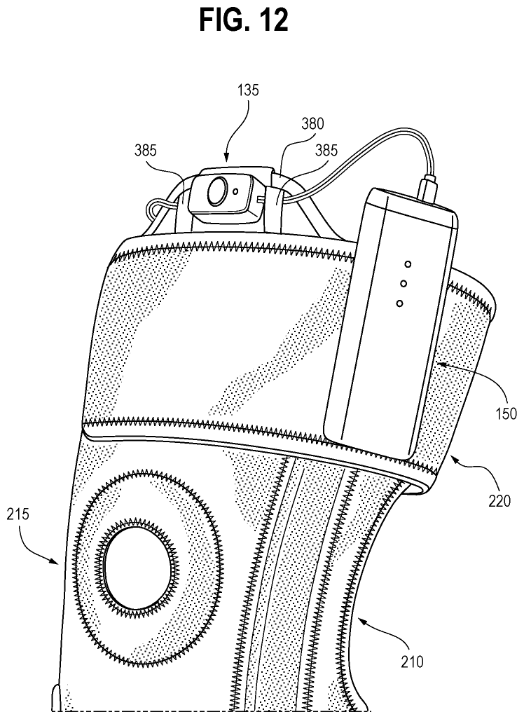

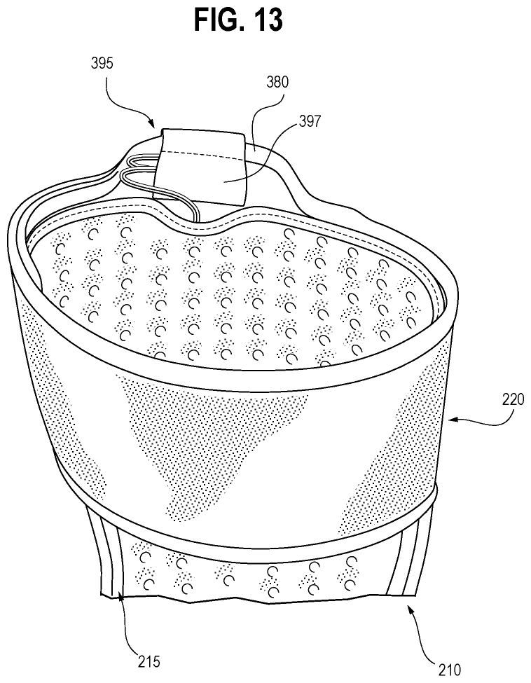

As illustrated in FIG. 12, the main body portion 215 of the brace 210 can also include a projection 380 that is sized and shaped to receive the controller 135. Releasable fasteners 385, such as loops, clips, hook and loop type fabric, or similar mechanisms, can releasably couple the controller 135 to the brace 210. This is particularly useful when the battery pack 150 is clipped onto or otherwise engaged with the brace 210. A cord management system 395, such as elastic loops 397, can also be used on the brace to manage the controller cords (FIG. 13).

From the foregoing, it will be seen that the various embodiments of the present invention are well adapted to attain all the objectives and advantages hereinabove set forth together with still other advantages which are obvious and which are inherent to the present structures. It will be understood that certain features and sub-combinations of the present embodiments are of utility and may be employed without reference to other features and sub-combinations. Since many possible embodiments of the present invention may be made without departing from the spirit and scope of the present invention, it is also to be understood that all disclosures herein set forth or illustrated in the accompanying drawings are to be interpreted as illustrative only and not limiting. The various constructions described above and illustrated in the drawings are presented by way of example only and are not intended to limit the concepts, principles, and scope of the present invention.

Many changes, modifications, variations, and other uses and applications of the present invention will, however, become apparent to those skilled in the art after considering the specification and the accompanying drawings. All such changes, modifications, variations, and other uses and applications which do not depart from the spirit and scope of the invention are deemed to be covered by the invention, which is limited only by the claims which follow.

What is claimed is:

1. A modular brace system comprising:
a knee or back brace comprising a main body portion and side straps configured to wrap around a body part of a user and releasably secure to another portion of the brace; and
at least one of a gel pack and a heat pack releasably securable to the brace via a plurality of hook and loop fasteners.

2. The modular brace system of claim 1, wherein gel in the gel pack is heatable and coolable.

3. The modular brace system of claim 1, wherein the heat pack comprises a plurality of electrical components configured to provide ambient heat through the heat pack.

4. The modular brace system of claim 1, further comprising a cord management system configured to manage a plurality of cords associated with the at least one of the gel pack and the heat pack.

5. The modular brace system of claim 1, wherein the main body portion is made of mesh fabric.

6. The modular brace system of claim 1, wherein the main body portion comprises a plurality of stabilization rods configured to provide support to the brace.

7. The modular brace system of claim 1, wherein the brace comprises a plurality of compression straps configured to provide compression to the brace.

8. The modular brace system of claim 1, wherein the main body portion tapers inwardly from a middle portion of the main body portion to end portions of the main body portion.

9. A modular brace system comprising:
a brace comprising a main body portion and side straps configured to wrap around a body part of a user and releasably secure to another portion of the brace; and
a gel pack releasably securable to the brace via a plurality of hook and loop fasteners.

10. The modular brace system of claim 9, wherein gel in the gel pack is heatable and coolable.

11. The modular brace system of claim 9, wherein the brace is used for at least one of a knee or a back.

12. The modular brace system of claim 9, further comprising a heat pack releasably securable to the brace.

13. The modular brace system of claim 12, wherein the heat pack comprises a plurality of electrical components configured to provide ambient heat through the heat pack.

14. The modular brace system of claim 12, wherein the heat pack is releasably securable to the brace via a plurality of hook and loop fasteners.

15. The modular brace system of claim 9, wherein the main body portion tapers inwardly from a middle portion of the main body portion to end portions of the main body portion.

16. A modular brace system comprising:
a brace comprising a main body portion and side straps configured to wrap around a body part of a user and releasably secure to another portion of the brace; and
at least one of a gel pack and a heat pack releasably securable to the brace via a plurality of hook and loop fasteners.

17. The modular brace system of claim 16, wherein gel in the gel pack is heatable and coolable.

18. The modular brace system of claim 16, wherein the heat pack comprises a plurality of electrical components configured to provide ambient heat through the heat pack.

19. The modular brace system of claim 16, wherein the brace is used for at least one of a knee or a back.

20. The modular brace system of claim 16, wherein the main body portion tapers inwardly from a middle portion of the main body portion to end portions of the main body portion.
